(19) United States
(12) Patent Application Publication
(10) Pub. No.: US 2016/0051402 A1
(43) Pub. Date: Feb. 25, 2016

(54) POST-SURGICAL ARTICLES FOR REDUCTION OF SWELLING, EDEMA, AND BRUISING

(71) Applicant: ALPS South, LLC, St. Petersburg, FL (US)

(72) Inventors: Aldo A. Laghi, St. Petersburg, FL (US); Edyta Hall, St. Petersburg, FL (US)

(21) Appl. No.: 14/602,893

(22) Filed: Jan. 22, 2015

Related U.S. Application Data
(60) Provisional application No. 62/062,372, filed on Oct. 10, 2014.
(60) Provisional application No. 62/051,847, filed on Sep. 17, 2014.
(60) Provisional application No. 62/040,771, filed on Aug. 22, 2014.

(51) Int. Cl. A61F 7/02

(52) U.S. Cl. CPC A61F 7/02; A61F 2007/0003

(57) ABSTRACT

Disclosed herein are garments having any combination of low thermal conductivity, high durability and resiliency, and with the optional ability to evenly distribute compressive forces. When compared to currently used compression garments, these compression garments advantageously result in better patient comfort and overall improved healing processes. In certain aspects, the disclosed compression garments include post-surgical treatment masks. For example, the post-surgical treatment mask may include a thermoformable assembly having a thermoformable resin arranged between inner and outer knitted fabric layers, the thermoformable assembly configured to be heated to a glass transition temperature of the thermoformable resin such that the thermoformable assembly can be shaped to conform to contours of a user's face, and a styrene-based polymeric gel layer arranged on the inner knitted fabric layer.

### CROSS-REFERENCE TO RELATED APPLICATIONS

This application claims priority to U.S. Provisional Patent Application No. 62/040,771 filed on Aug. 22, 2014, U.S. Provisional Patent Application No. 62/051,847 filed on Sep. 17, 2014, and U.S. Provisional Patent Application No. 62/062,372 filed on Oct. 10, 2014, the contents of which are incorporated by reference herein in their entirety.

### TECHNICAL FIELD

The present invention relates to the field of articles used to reduce post-surgical swelling and bruising by applying thermal therapy (e.g., cold compression) for extended periods of time to a surgical site.

### BACKGROUND OF THE INVENTION

Following non-elective and elective surgical procedures, it is known that surgical sites and areas surrounding surgical sites bruise, swell, and often times have large amounts of localized edema due to the high amount of trauma incurred during these procedures. Although bruising, swelling, and edema naturally occur in response to trauma and are associated with natural, biological healing processes, excessive bruising, swelling, and edema may impede healing, and in certain instances, excessive bruising, swelling, and edema may even lead to post-surgical complications including, but not limited to, blood clot formation, embolism(s), and/or thrombosis.

Various types of post-surgical garments are currently used to treat surgical sites in an attempt to reduce bruising, swelling, and edema sometimes associated with surgery. For example, most post-surgical garments (e.g., compression garments) utilize only fabric and/or hydrogels, which are used in an attempt to reduce and/or prevent post-surgical bruising, swelling, and edema. Post-surgical garments that incorporate thermal therapy devices often utilize hydrogels because they are relatively inexpensive, easy to manufacture, and may be easily adapted for the purposes of compression therapy, thermal therapy, cold compression therapy, or any combination thereof.

Although conventional post-surgical procedures utilize hydrogel compression garments, numerous problems exist with these compression garments. First, hydrogels (i.e., water-based gels) have a high heat capacity and high thermal conductivity due to their high water content, which means that these gels draw heat very quickly due to their high thermal conductivity and for a long time due to their high heat capacity. When used during thermal therapy and cold compressive therapy, the hydrogel's high heat capacity and high thermal conductivity may create an initial and maintained shock at an application site because the hydrogel tends to draw heat very quickly and for a very long time at the application site. Because of hydrogel's high thermal conductivity and high heat capacity, a patient may experience much discomfort, including damaged skin and/or additional tissue(s), when a hydrogel based compression garment is applied to a post-surgical site for the purposes of thermal therapy or cold compression therapy. Second, hydrogels and hydrogel based compression garments typically lack adequate stretching properties to evenly distribute compressive forces around the surgical site, which disadvantageously leads to reduced treatment efficacy. For example, these uneven compressive forces result in random application of high compressive pressure and low compressive pressure around a surgical site. Areas receiving too much pressure (i.e., sites having high compressive pressure) often result in patient discomfort and potentially diminished vascular function, which may further lead to skin damage and other tissue damage around the surgical site. Areas receiving too little pressure may lead to edema micro-pooling at the surgical site, which may subsequently lead to delayed healing and/or further tissue damage. Third, hydrogels often lack durability because of the high water content within these materials. Specifically, these hydrogels lack tear strength due to high water content, which makes them prone to tearing during handling and application. Hydrogels also tend to desiccate very quickly during usage. Thus, for at least the above discussed reasons, hydrogels and hydrogel garments including hydrogel compression garments have very short lifespans for usage coupled with undesirable treatment results.

### BRIEF SUMMARY OF THE INVENTION

Therefore, a need exists to provide garments having low thermal conductivity, low heat capacity, and high durability and resiliency when worn by a wearer. In certain aspects, these garments further include compression garments having the ability to provide evenly distributed compressive forces (e.g., a high modulus of elasticity). Disclosed herein are garments (e.g., compression garments) having low thermal conductivity, low heat capacity, and high durability and resiliency, which overcome the disadvantages associated with conventional hydrogels and hydrogel garments and compression garments. In certain aspects, the garments further include the ability to provide evenly distributed compressive forces (e.g., a high modulus of elasticity). Furthermore, these garments advantageously result in better patient comfort and overall improved healing processes due to the combination of any of the following features: (i) low thermal conductivity, (ii) low heat capacity, (iii) high durability and resiliency, and (iv) formability. In certain aspects, the disclosed garments include compression garments that evenly distribute compressive forces.

The disclosed garments may include a post-surgical treatment mask including a thermoformable assembly having a thermoformable resin arranged on a knitted fabric layer, the thermoformable assembly configured to be heated to a glass transition temperature of the thermoformable resin such that the thermoformable assembly can be shaped to conform to contours of a user's face, and a styrene-based polymeric gel layer arranged on the thermoformable assembly. In certain aspects, the disclosed garments may include a post-surgical treatment mask including a thermoformable assembly having a thermoformable resin arranged between inner and outer knitted fabric layers, the thermoformable assembly configured to be heated to a glass transition temperature of the thermoformable resin such that the thermoformable assembly can be shaped to conform to contours of a user's face, and a styrene-based polymeric gel layer arranged on the inner knitted fabric layer. This thermoformable resin should be configured to form a rigid structure that is capable of supporting and applying pressure to the gel when secured to the wearer.

In certain aspects, the thermoformable resin comprises a low-melt co-polyester, a poly-caprolactone, or a combination thereof.

In certain aspects, the thermoformable assembly is configured to be heated to a temperature of no more than 120° F. above the glass transition temperature of the thermoformable resin.

In certain aspects, the inner and outer knitted fabric layer are the same.

In certain aspects, the inner and outer knitted fabric layer are different.

In certain aspects, the knitted outer layer fabric is made with a non-low melt polyester yarn, a non-low melt nylon yarn, a non-low melt polyproprylene yarn, a non-low melt polyethylene yarn, cotton yarn, wool yarn, any combinations thereof, and these yarns may be either multifilament or monofilament. In certain aspects, the yarns included within the outer layer knitted fabric layers are multifilament having a mass ranging from 110 to 160 denier, and the knit fabric of the outer fabric layers is a weft knit having multidirectional stretch characteristics that aid in further enhancing durability of the thermoformable assembly. In certain aspects, the knit fabric of the inner and outer fabric layers independently have an elasticity ranging from 80 to 140% in a vertical direction and from 60 to 100% in the horizontal direction.

In certain aspects, the knitted inner layer fabric is made with a non-low melt polyester yarn, a non-low melt nylon yarn, a non-low melt polyproprylene yarn, a non-low melt polyethylene yarn, cotton yarn, wool yarn, any combinations thereof, and these yarns are preferably multifilament the knit fabric of the outer fabric layers is a weft knit having multidirectional stretch characteristics that aid in further enhancing durability of the thermoformable assembly while concurrently enhancing the effects of post-surgical treatment (i.e., reducing edema, swelling, and/or bruising).

In certain aspects, the styrene-based gel is removable from the thermoformable assembly.

In certain aspects, the stryene-based gel is from 0.050 inches to 0.625 inches, and more preferably 0.100 inches to 0.300 inches in thickness.

In certain aspects, the styrene-based gel has a thermal conductivity ranging from 0.05 to 3.00 W/mk.

In certain aspects, the mask includes a strap configured to secure around a user's head to hold the mask in place on the user's face.

In certain aspects, the mask includes hook and loop fasteners for securing the strap to the mask.

In certain aspects, the mask includes two straps configured to secure around a user's head to hold the mask in place on the user's face.

In certain aspects, the mask is a partial face mask adapted to cover a user's eyes and the bridge of the nose.

In another embodiment, the garment may include a post-surgical face mask made from a styrene-based gel shaped to conform to and cover a wearer's face including a chin, a nose, cheeks, forehead, and ears, the mask having openings at the wearer's eyes, nose, and mouth; and a strap configured to fasten around the forehead and under the chin to secure the mask in place.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention, and are intended to provide an overview or framework for understanding the nature and character of the invention as it is claimed. The accompanying drawings are included to provide a further understanding of the invention, and are incorporated in and constitute a part of this specification.

### BRIEF DESCRIPTION OF THE DRAWINGS

These and other features, aspects and advantages of the present invention are better understood when the following detailed description of the invention is read with reference to the accompanying drawings, in which:

FIG. 1 depicts a front view of the post-surgical treatment mask according to the first embodiment being worn by a wearer;

FIG. 2 depicts a cross section of the layers included within the post-surgical treatment mask according to the first embodiment;

FIG. 3 depicts an exploded view of the layers included within the post-surgical treatment mask according to the first embodiment;

FIG. 4 depicts a back view of the post-surgical treatment mask according to the first embodiment;

FIG. 5 depicts a front view of the post-surgical treatment mask according to the first embodiment;

FIG. 6 depicts a front view of the post-surgical treatment mask according to the first embodiment including straps and hook and loop fasteners;

FIG. 7 depicts a back view of the post-surgical treatment mask according to the first embodiment including straps and hook and loop fasteners;

FIG. 8 depicts a front view of the mask according to a second embodiment of the invention;

FIGS. 9(a) and 9(b) depict multiple side views of the mask according to a second embodiment of the invention;

FIG. 10 depicts a garment according to another embodiment including the styrene based gel permanently positioned on an elongate, elastic bandage and adapted for application to a wearer's abdominal area;

FIGS. 11 and 12 depict multiple views of a garment according to another embodiment including the styrene based gel permanently positioned on an elongate, elastic bandage and adapted for application to a wearer's chin;

FIG. 13 depicts another embodiment including a styrene based gel adapted to conform to a wearer's breast;

FIG. 14 depicts a top view of another embodiment of the post-surgical treatment mask;

FIG. 15 depicts a perspective view of the post-surgical treatment mask of FIG. 14;

FIGS. 16(a) and 16(b) depict another embodiment of the post-surgical mask having a thermoformable assembly and the styrene based gel; FIG. 16(b) further depicts a cross section of this embodiment showing the knitted outer layer, the thermoformable resin layer, and the styrene based gel positioned on the thermoformable resin layer;

FIGS. 17(a) and 17(b) depict various perspective views of the post-surgical mask of FIG. 14; and

FIG. 18 depicts a garment including the styrene based gel positioned on an elongate, elastic bandage and adapted for application to a wearer's chin and neck.

### DETAILED DESCRIPTION OF THE INVENTION

The present invention will now be described more fully hereinafter. It is to be understood that the aspects described below are not limited to specific compounds, synthetic methods, or uses as such may, of course, vary. It is also to be understood that the terminology used herein is for the purpose of describing particular aspects only and is not intended to be limiting. However, the invention may be embodied in many different forms and should not be construed as limited to the representative embodiments set forth herein. The exemplary embodiments are provided so that this disclosure will be both thorough and complete, and will fully convey the scope of the invention and enable one of ordinary skill in the art to make, use and practice the invention.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

It is understood that any given particular aspect of the disclosed compositions and methods can be easily compared to the specific examples and embodiments disclosed herein. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

Disclosed herein are garments having low thermal conductivity and high durability and resiliency. In certain aspects, the disclosed garments include compression garments configured to provide evenly distributed compressive forces (e.g., a high modulus of elasticity). When compared to currently used post-surgical garments, these garments (e.g., compression garments) advantageously result in better patient comfort and overall improved healing processes due to the combination of any of the following: (i) low thermal conductivity, (ii) high durability and resiliency, and in certain aspects (iii) evenly distributed compressive forces.

Each of the garments include a polymeric gel material, with the proviso that this gel material preferably is not a hydrogel. In other words, water content is highly limited within the gel (e.g., including water amounts of less than 1 wt %, 0.5 wt %, 0.3 wt %, or 0.1 wt % of the overall gel), or water, minus any impurities, may not be added to or within the disclosed polymeric gel. When compared with hydrogels, in certain aspects the disclosed gels preferably have lower thermal conductivity than hydrogels (and high heat capacity in certain aspects) so the disclosed gels draw less heat at a lower rate than hydrogels. These properties are preferable within the disclosed articles because the disclosed gels achieve better patient comfort and overall improved healing processes at an application site. In certain aspects, this gel material is made from a triblock copolymer and plasticizing oil and optionally includes one or more additives. However, to potentially reduce manufacturing costs and in certain preferred aspects, the gel material may only include the disclosed triblock copolymer(s), plasticizing oil, and optionally additives.

In certain aspects, triblock copolymer is a styrene-based polymer that includes styrene and at least one of ethylene, butadiene, butylene, propylene, or isoprene, for example a styrene-ethylene-ethylene-propylene-styrene, a styrene-ethylene-butylene-styrene, or a styrene-ethylene-propylene-styrene. In certain embodiments, thermoplastic elastomers according to the present invention comprises a polymer that is a hydrogenated poly(styrene-b-isoprene), a hydrogenated poly(styrene-b-isoprene-b-styrene), a hydrogenated poly(styrene-b-butadiene-b-styrene), a hydrogenated poly(styrene-b-isoprene/butadiene-b-styrene), or combinations thereof. In certain embodiments, the thermoplastic article comprises any of polystyrene-b-poly(ethylene/propylene) (SEP), polystyrene-b-poly(ethylene/propylene)-b-polystyrene (SEPS), polystyrene-b-poly(ethylene/butylene)-b-polystyrene (SEBS), or polystyrene-b-poly(ethylene-ethylene/propylene)-b-polystyrene (SEEPS), or any combination thereof. In certain aspects, the amount of triblock copolymer included within the gel material ranges from 50 to 500 parts per hundred, 75 to 300 parts per hundred, 90 to 200 parts per hundred, or 95 to 120 parts per hundred. In preferred aspects, the triblock copolymer is 100 parts per hundred and all other disclosed components (e.g., plasticizer/plasticizing oil, antioxidant, other additives) are weighed/weighted against the triblock copolymer. For example, if a 1:1.2 ratio of triblock copolymer to plasticizer is desired, this would be measured as 100 parts triblock copolymer and 120 parts plasticizer.

In certain aspects, the disclosed gels are made with and/or include one or more plasticizing oils that may include, for example, a paraffinic oil, naphtenic oil, a mineral oil, or a synthetic liquid oligomer of a polybutene, a polypropene, or a polyterpene oil. Suitable oils include plasticizing oils such as paraffinic oils, naphtenic petroleum oils, petroleum mineral oils, and synthetic liquid oligomers of polybutene, polypropylene, polyterpene, etc. may be used. In preferred aspects, the plasticizer is petroleum mineral oil with a viscosity of 10 to 20 centistokes, more preferably 11 to 17 centistokes, and most preferably 11 to 14 centistokes. In certain aspects, the plasticizing agent used within the gel includes petroleum mineral oil is 12 centistokes. The amount of plasticizer included within the gel ranges from 200 to 1000 parts per hundred, 250 to 750 parts per hundred, preferably 300 to 800 parts per hundred, more preferably 400 to 700 parts per hundred, and most preferably 500 to 650 parts per hundred. In certain aspects, the oil may also be seeded with an insoluble fine powder such as talc.

The oil or other plasticizing agent (also referred to herein as a plasticizer) can be added to the triblock copolymer in order to obtain the desired mechanical properties, such as elasticity, softness (or hardness), thermal conductivity, and elongation, tear and tensile strength characteristics of the resulting gel. For example, in some embodiments, suitable mechanical properties of the resulting gel include: (a) hardness between approximately 10 to 70 durometer on the Shore 00 scale, between 15 to 60 durometer on the Shore 00 scale, and more preferably about 15 to 40 durometer on the Shore 00 scale; (b) ultimate elongation of approximately 300 to 2000 percent, more preferably about 1500 percent; (c) tensile modulus at 300 percent elongation of between about 5 to 300 psi, more preferably about 30 psi; and/or (d) a thermal conductivity of 0.05 to 3.00 W/mk, 0.1 to 1.5 W/mk.

In certain embodiments, the disclosed gels may be manufactured by mixing together the styrene-based polymer, the plasticizing oil(s), and one or more optional additives, e.g., an antioxidant, an antimicrobial agent, and/or other additives, to form a mixture which is melted then cooled into the resulting gel. The additives may include, for example, 0.5 to 20 parts, 1 to 12 parts, or 1.5 to 8.5 parts. The antioxidants may include a phenolic antioxidant. For example, the phenolic antioxidant may include at least one of isobutylenated methylstyrenated phenol, a styrenated phenol, various o-, m-, p-cresols (e.g., 4,4′thiobis-6-(t-butyl-m-cresol), 4,4′-butylidenebis-b-(t-butyl-m-cresol)), 2,6-di-tert-butyl-p-cresol, (octadecanoxycarbonylether) phenol, tetrakis-(methylene-(3,5-ditertbuty-4-hydrocinnamate)methane, 2,2′-methylenebis(4-methyl-6-nonyl) phenol, 1,3,5-tris(3,5-di-tert-butyl-4-hydroxybenxyl+1,3,5-triazine-2,4,6 (1H,3H,5H)-trione, or any combination thereof, which may each independently range from 1 to 8 parts per hundred or 2 to 5 parts per hundred or in combination may range from 1 to 8 parts per hundred or 2 to 5 parts per hundred. The antimicrobial agents may include, for example, at least one of silver zeolite, silver zirconium phosphate, silver nitrate, silver thiosulfate, silver sulphadiazine, silver fusidate, and quaternary ammonium compounds (QAC). Other classes of silver-based antimicrobial agents may be used as well, for example a silver acetate, a silver bromide, a silver carbonate, a silver chlorate, a silver chloride, a silver citrate, a silver fluoride, a silver iodate, a silver lactate, a silver nitrate, a silver nitrite, a silver perchlorate or a silver sulfide. In addition, one or more other antimicrobial agents may be used in conjunction with or instead of such silver-based antimicrobial agents. When present, the antimicrobial agent, may only include from 0.3 to 1 wt % or from 0.5 to 0.9 wt % of the overall weight of the gel. In certain aspects, the antimicrobial agent may range from 50 to 200 parts per hundred, preferably 65 to 175 parts per hundred, and most preferably 80 to 150 parts per hundred. These antioxidants and antimicrobial agents may be included within the gel to further enhance the therapeutic purposes of the resulting gel and embodiments disclosed herein. For example, in certain aspects, the antioxidants and/or antimicrobial agents may seep out from the gel onto the wearer's skin and/or bandages. In theory, the antioxidants and/or antimicrobial agents that seep from the gel may contact the surgical site or areas around the surgical site, thus resulting in beneficial delivery of these antioxidants and/or antimicrobial agents that further aid in improved and expedited healing of the surgical site(s).

In alternative aspects, one or more of the above discussed additives may be added to the mixture after the mixture is melted or during the cooling process. After heating and mixing the mixture of styrene-based polymer, the plasticizing oil(s), and one or more optional additives, these components are melted together in such a manner that a homogeneous, molten mixture is obtained. After obtaining the molten mixture, the molten mixture is extruded via an extruder, molded via a molding machine, or other similar heated vessel into the desired shapes and thickness. In certain aspects and when the disclosed gels are included in the disclosed garments and have a thickness ranging from 0.050 inches to 0.625 inches, more preferably 0.0625 inches to 0.400 inches in thickness, and most preferably 0.1 inches to 0.3 inches. These thicknesses are desired to ensure that maximum reduction and/or prevention of post-surgical, excessive bruising, swelling, and edema associated with surgical procedures.

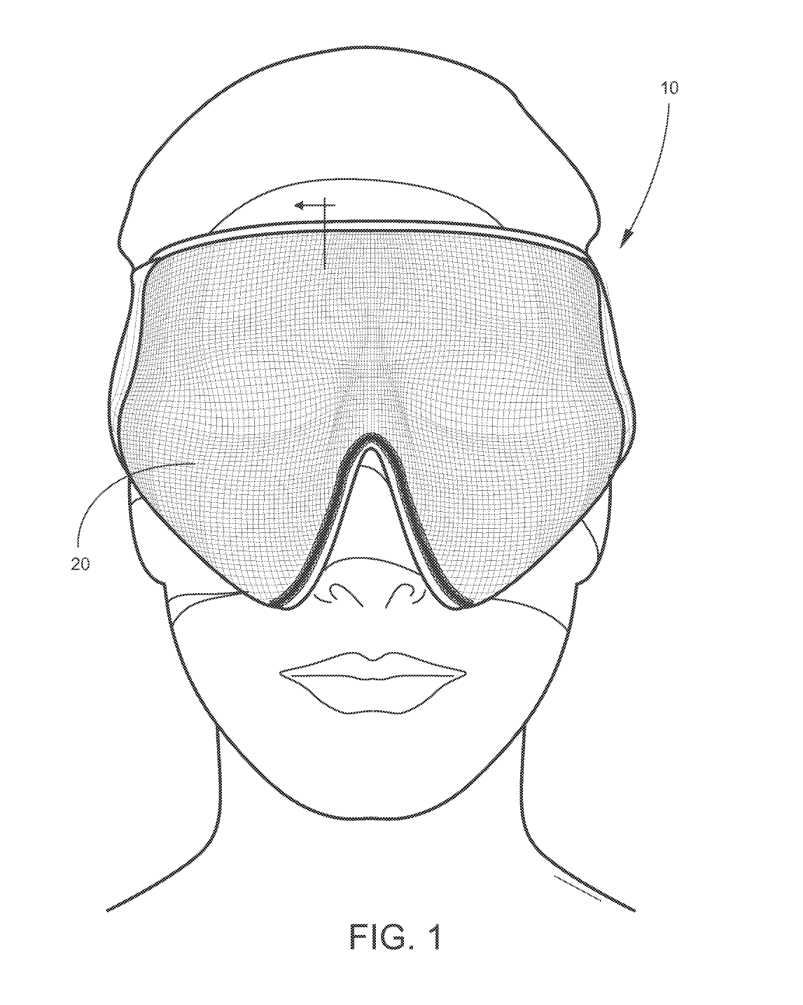

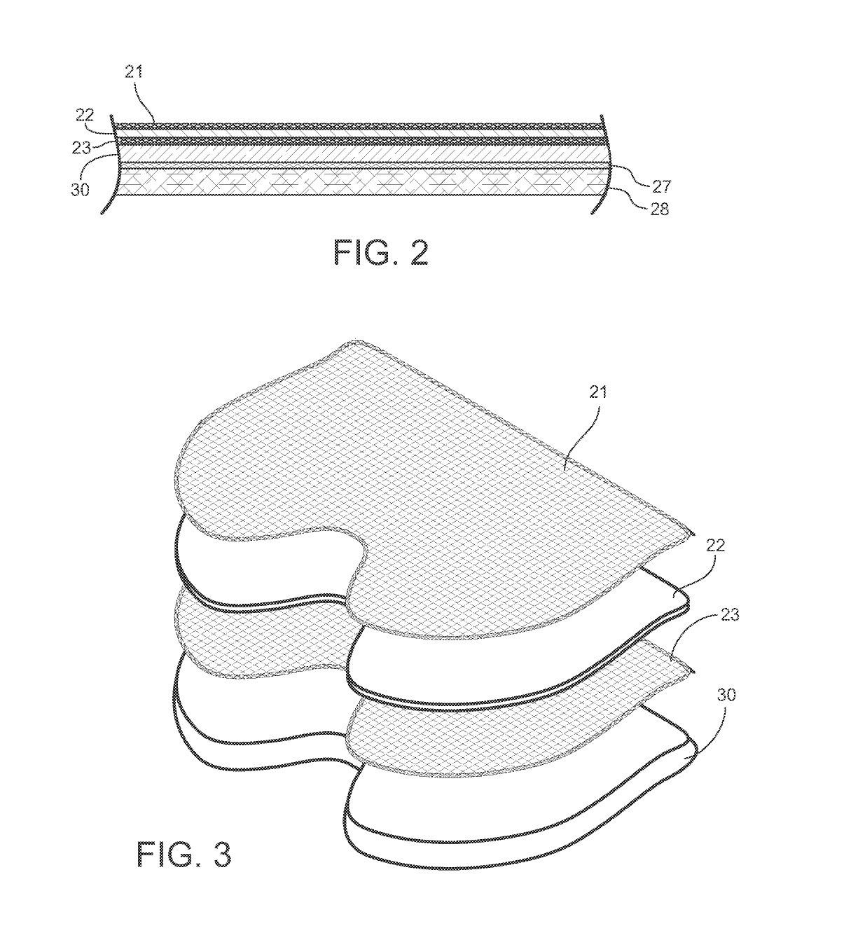

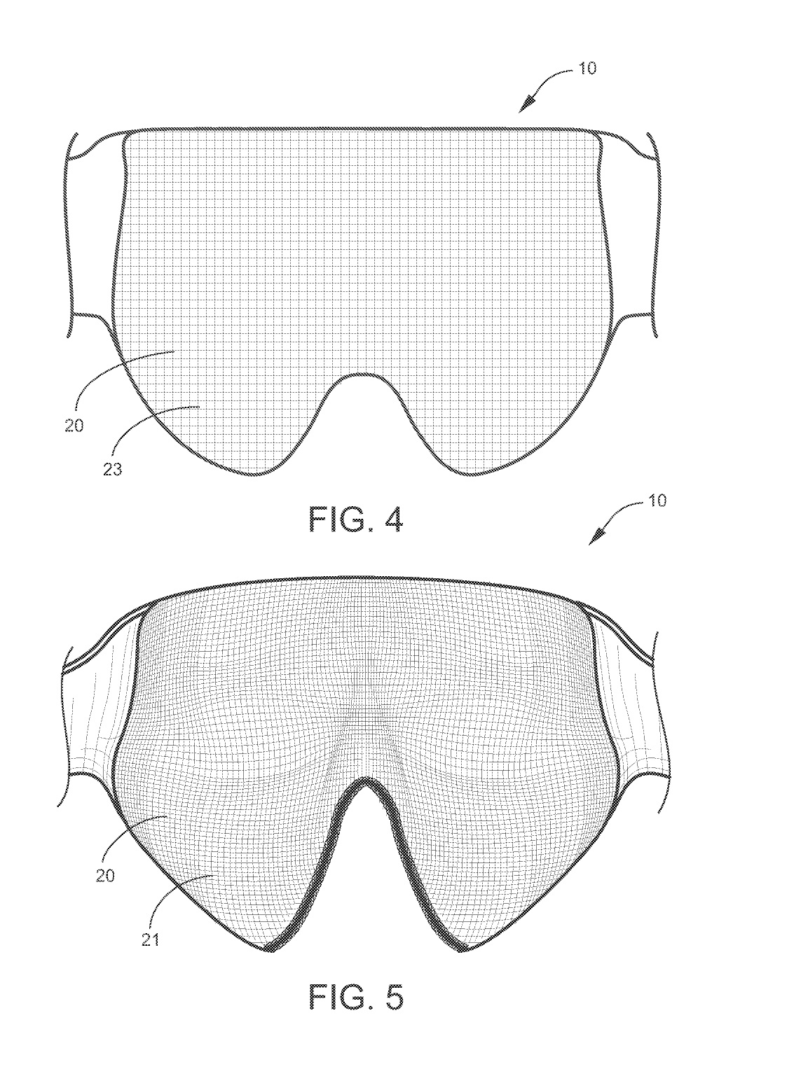

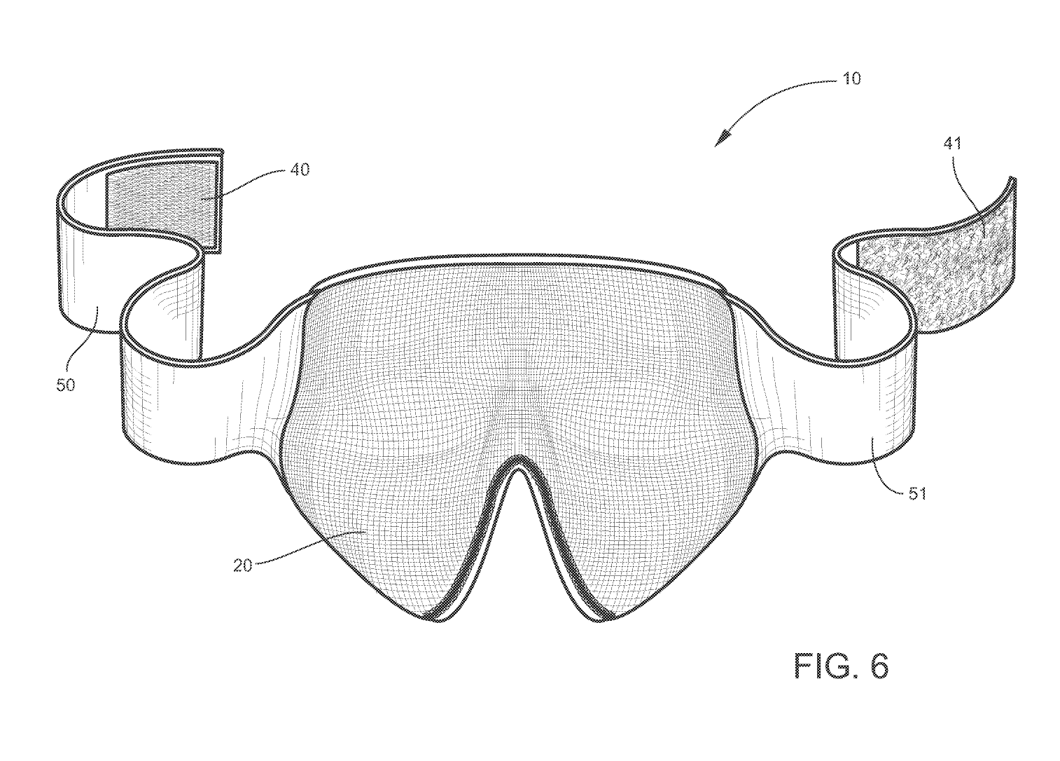

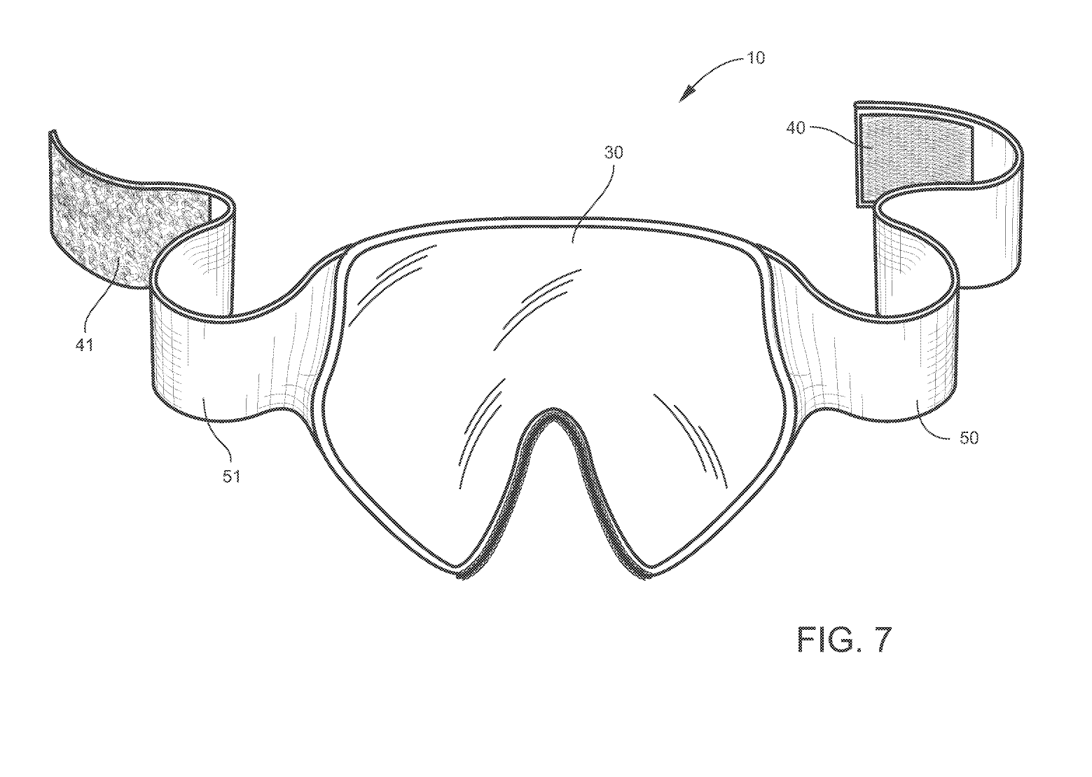

The disclosed garments will now be described in more detail with reference to the Figures. FIGS. 1-7 disclose a post-surgical treatment mask 10 according to a first embodiment including a thermoformable assembly 20 comprising a thermoformable resin 22 arranged between inner 23 and outer 21 knitted fabric layers, the thermoformable assembly configured to be heated to a glass transition temperature of the thermoformable resin such that the thermoformable assembly can be shaped to conform to contours of a user's face. In certain aspects, inner layer 23 is optional and may not be present. In this aspect, the post-surgical mask 10 also includes a styrene-based polymeric gel layer 30 arranged on the inner knitted fabric layer 23, or when inner knitted fabric layer 23 is not present, gel layer 30 may be arranged directly on thermoformable resin 22. As depicted in FIG. 2, when in use, gel layer 30 of post-surgical treatment mask 10 is placed on bandage 27, which directly covers flesh and/or post-surgical site 28. However, in certain aspects, the gel layer 30 is configured to directly contact the wearer's skin/flesh.

In certain aspects, the thermoformable resin includes at least one of a co-polyester, a poly-caprolactone, nylon, polypropylene, polyethylene, or a combination thereof. In certain aspects, the thermoformable resin is a low melt resin configured to begin melting or completely melt at temperatures ranging from 100° F. to 300° F., 120° F. to 250° F., 120° F. to 200° F., 120° F. to 150° F., 130° F. to 175° F., 130° F. to 150° F., 135° F. to 145° F., 140° F. to 160° F. In certain aspects, the thermoformable assembly is configured to be heated to a temperature of no more than 5° F., 10° F., 20° F., 35° F., 50° F., 65° F., 80° F., 100° F., or 120° F. above the glass transition temperature of the thermoformable resin. The thermoformable resin may initially include a knitted body made from a low-melt co-polyester yarn (e.g., having a melting temperature of under 200° F._), a poly-caprolactone yarn, nylon yarn, polypropylene yarn, polyethylene yarn, or a combination thereof. In certain aspects, the thermoformable resin and/or the knitted body that becomes the thermoformable resin may include any combination of a low-melt co-polyester yarn, a poly-caprolactone yarn, nylon yarn, polypropylene yarn, and polyethylene yarn and a polyester fiber (having a melting temperature above 300° F. and ranging from 300° F. to 500° F., preferably 300° F. to 400° F.). These low-melt yarns may preferably have a mass ranging from 110 to 160 denier, 120 to 150 denier, 130 to 145 denier, and 135 to 145 denier. This knitted body is preferably knittable/made on traditional flatbed or circular knitting machinery.

In certain aspects, the inner and outer layer knitted fabric layers 21, 23 are selected to further synergistically maximize the reduction and/or prevention of post-surgical, excessive bruising, swelling, and edema associated with surgical procedures. For example, these knitted fabric layers and the yarns included in the fabric layers and the filaments included within the yarns of the knitted layers may be selected to maximize compressibility of the gel to further maximize post-surgical treatment for the reduction and/or prevention of bruising, swelling, and edema. Depending on the desired effects, the inner and outer knitted fabric layer are the same, or alternatively, the inner and outer knitted fabric layer are different. For example, the knitted inner and outer layer fabric layers may independently be made from, for example, a non-low melt polyester yarn, a non-low nylon yarn, a non-low polyproprylene yarn, a non-low melt polyethylene yarn, cotton yarn, wool yarn, any combinations thereof, and these yarns may be either multifilament or monofilament. In certain aspects, the yarns included within the inner and outer layer knitted fabric layers are multifilament having a mass ranging from 110 to 160 denier, 120 to 150 denier, 130 to 145 denier, and 135 to 145 denier. In certain aspects, the knit fabric of the inner and outer fabric layers are independently a weft knit having multidirectional stretch characteristics that aid in further enhancing durability of the thermoformable assembly. The knit fabric of the inner and outer fabric layers may independently include from 6 to 12 courses per cm², more preferably from 8 to 10 courses per cm² and from 9 to 14 wales per cm², more preferably 10 to 12 wales per cm² in the weft knit. In certain preferred aspects, the inner and outer fabric layers independently include 9 courses and 11 wales per cm². In certain aspects, the knit fabric of the inner and outer fabric layers independently have an elasticity ranging from 80 to 140%, preferably 100 to 130%, and most preferably 115 to 125% in a vertical direction and from 60 to 100%, preferably 70 to 90%, and most preferably 75 to 85% in the horizontal direction. In certain aspects, the inner layer 21, outer layer 23, and the knitted body configured to be the thermoformable resin may independently include a milano knitting pattern, a plain jersey knitting pattern, an interlock jersey knitting pattern, an interlock knitting pattern, a rib knitting pattern, a ponte de roma knitting pattern, or any combination thereof.

When initially making the thermoformable assembly, the knitted body made from a co-polyester yarn, a poly-caprolactone yarn, or a combination thereof and optionally having a polyester fiber may be positioned in between the inner and outer layer fabric layers. After layering this stack, this layered stack is heated to a sufficient temperature to melt the knitted body from a co-polyester yarn, a poly-caprolactone yarn, or a combination thereof to bond these layers together. In certain aspects and because the knitted body from a co-polyester yarn, a poly-caprolactone yarn, or a combination thereof includes fabric “windows”, this knitted body of co-polyester yarn, a poly-caprolactone yarn, or a combination thereof may be preferred over a solid sheet (e.g., a laminate layer) made from the same material because the windows and knitted structure may allow for stronger bonding while concurrently lowering manufacturing costs of the thermoformable assembly. However, in certain alternative aspects, a solid sheet of the thermoformable resin may be used when manufacturing the thermoformable assembly. In additional aspects, the thermoformable assembly is preferably configured to be repeatedly heated and reshaped to the user's contours. For example, with regard to a rhinoplasty, the thermoformable assembly may be heated to or slightly above the thermoformable resin's glass transition temperature to ensure that the thermoformable assembly may be shaped to the user's contours. It is important that thermoformable resin hardens and becomes rigid after heating to (or beyond) its glass transition temperature. However, it is also important that the thermoformable resin does not become brittle after heating such that the thermoformable assembly is fragile, lacks durability, and may not be re-heated and/or re-shaped. Thus, the disclosed thermoformable assembly may be advantageously repeatedly heated and re-shaped into a rigid structure having desired contours.

The styrene-based gel is used in combination with the thermoformable assembly, and in certain aspects, the styrene-based gel is removable from the thermoformable assembly. However, in other aspects, the styrene-based gel is permanently attached to the thermoformable assembly. In each of these aspects, the stryene-based gel has a thickness ranging from 0.05 inches to 0.625 inches, more preferably 0.1 inches to 0.5 inches, and most preferably 0.250 to 0.50 inches to further ensure maximum reduction and/or prevention in post-surgical bruising, swelling, and edema.

The mask further includes a strap 50, 51 configured to secure around a user's head to hold the mask in place on the user's face, and the mask includes hook and loop fasteners 40, 41 for securing the strap to the mask. In certain aspects, the hook or loop fastener may be attached to one end of the strap and the complimentary hook or loop fastener may be attached on the thermoformable assembly 20 or on another strap. For example, in certain aspects, the mask includes two straps 50, 51 configured to secure around a user's head to hold the mask in place on the user's face.

The post-surgical treatment mask 10 may preferably be a partial face mask configured for placement over a user's eyes and the bridge of the nose. The styrene based gel of this partial face mask can be heated and/or cooled to a desired temperature and applied and fastened to the user's face to treat post-surgical bruising, swelling, and edema associated with, for example, rhinoplasty, eyelid surgery, check implantation, or any combination thereof. This post-surgical treatment mask 10 is preferred over conventional hydrogel masks because unlike hydrogel based masks, this mask exhibits low thermal conductivity and high durability and resiliency coupled with the ability to provide evenly distributed compressive forces (e.g., a high modulus of elasticity). Additionally, this mask 10 advantageously results in better patient comfort and overall improved healing due to the combination of any of (i) low thermal conductivity, (ii) high durability and resiliency, (iii) evenly distributed compressive forces, and (iv) customizability or formability achieved by post-surgical treatment mask 10. In certain aspects, post-surgical mask 10 further includes, for example, fabric or a fabric layer that lines the peripheral edges of mask 10 and conceals one or more layers of the thermoformable assembly. For example, in certain aspects, the fabric or fabric layer completely lines the peripheral edges of mask 10 and completely conceals the thermoformable assembly such that the thermoformable assembly is not visible to the wearer or another observer. In certain aspects, mask 10 is more aesthetically pleasing when the fabric or fabric layer lines the peripheral edges of mask 10.

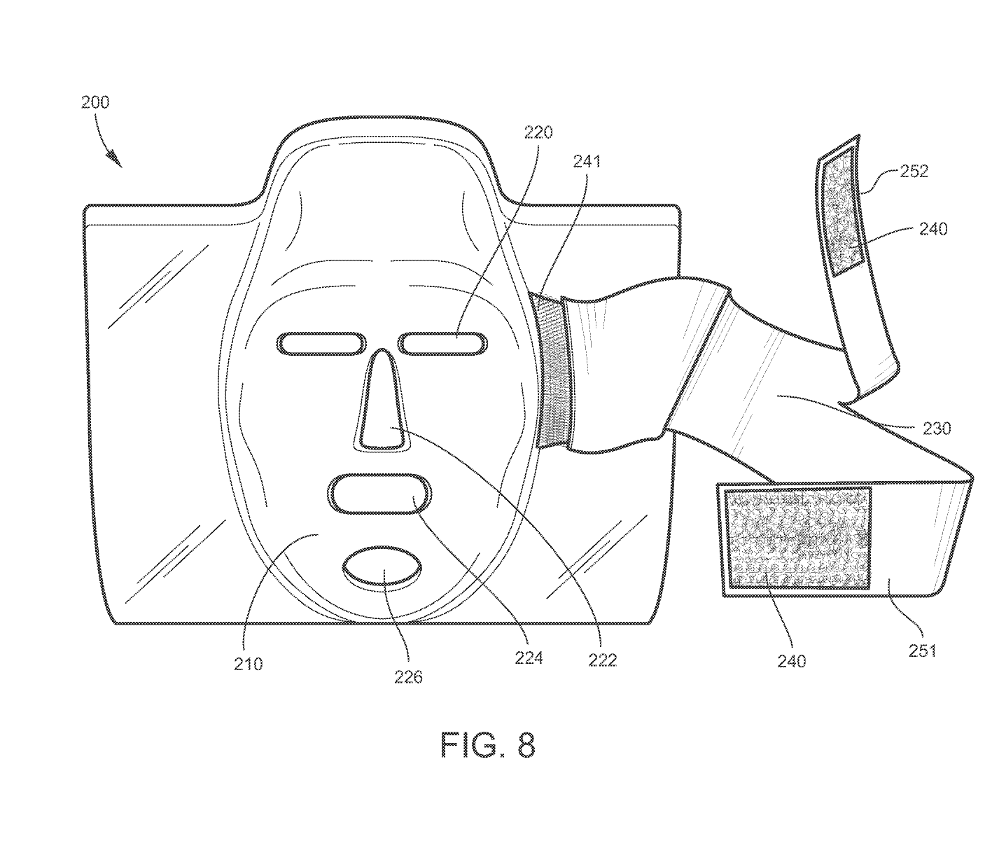

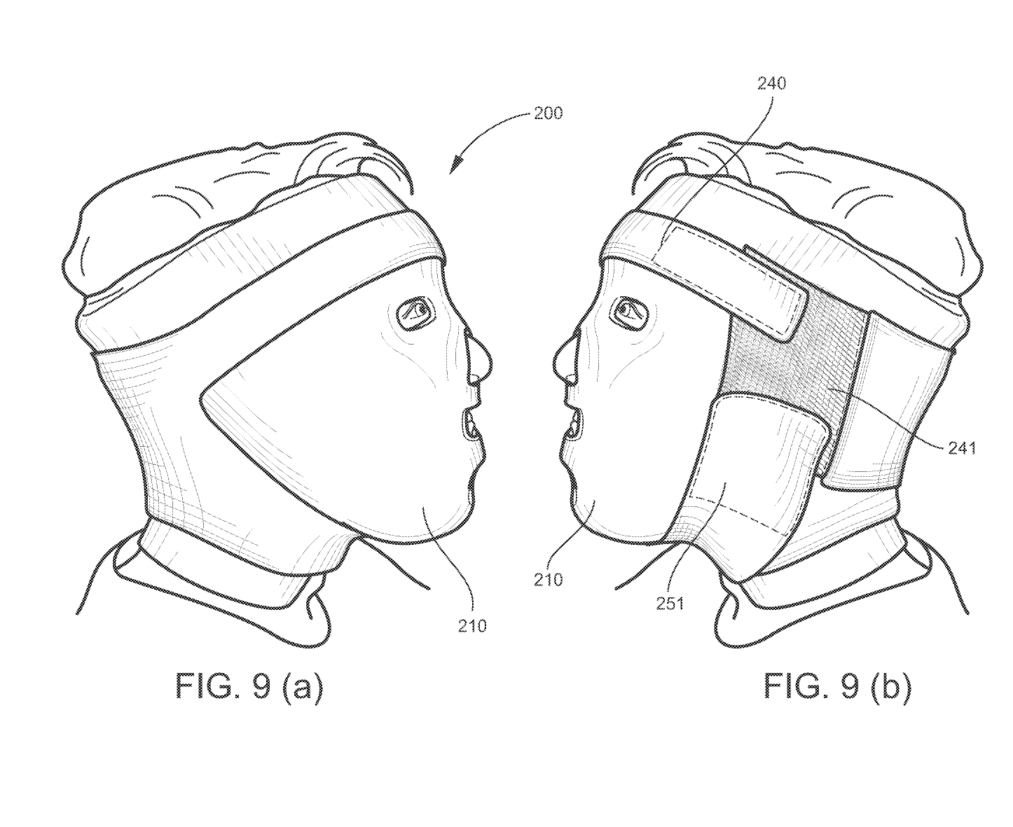

In another embodiment and as further illustrated in FIGS. 8, 9(a), and 9(b), the garment may include a post-surgical face mask 200 including a mask 210 made from a styrene-based gel shaped to conform to and cover a wearer's face including a chin, a nose, cheeks, forehead, and ears, the mask having openings at the wearer's eyes 220, nose 220, mouth 224, and optionally the chin 226; and a strap 230 configured to fasten around the forehead and under the chin to secure the mask in place. For example, this post-surgical face mask 200 may consist essentially of or consist only of the styrene-based gel, which conforms to and covers a wearer's face including a chin, a nose, cheeks, forehead, and ears, the mask having openings at the wearer's eyes, nose, and mouth, and a strap 230 configured to fasten around the forehead and under the chin to secure the mask in place. In certain aspects, the strap includes complimentary hook and look fasteners 240, 241 on opposite ends of the strap. One end of the strap may be bifurcated such that a portion of the bifurcated strap 251 wraps around the chin of a user while the other portion of the bifurcated strap 252 is configured to wrap around the head of the user in such a manner that the mask may be securely fastened to a user with the strap. In certain aspects, the strap is an elastic strap configured to stretch and apply compressive forces when securely fastening the mask to the user. As illustrated in FIGS. 8, 9(a), and 9(b) the strap 230 may be attached to, for example, on the outermost surface of the gel, or in certain aspects, the strap 230 may be positioned within the gel.

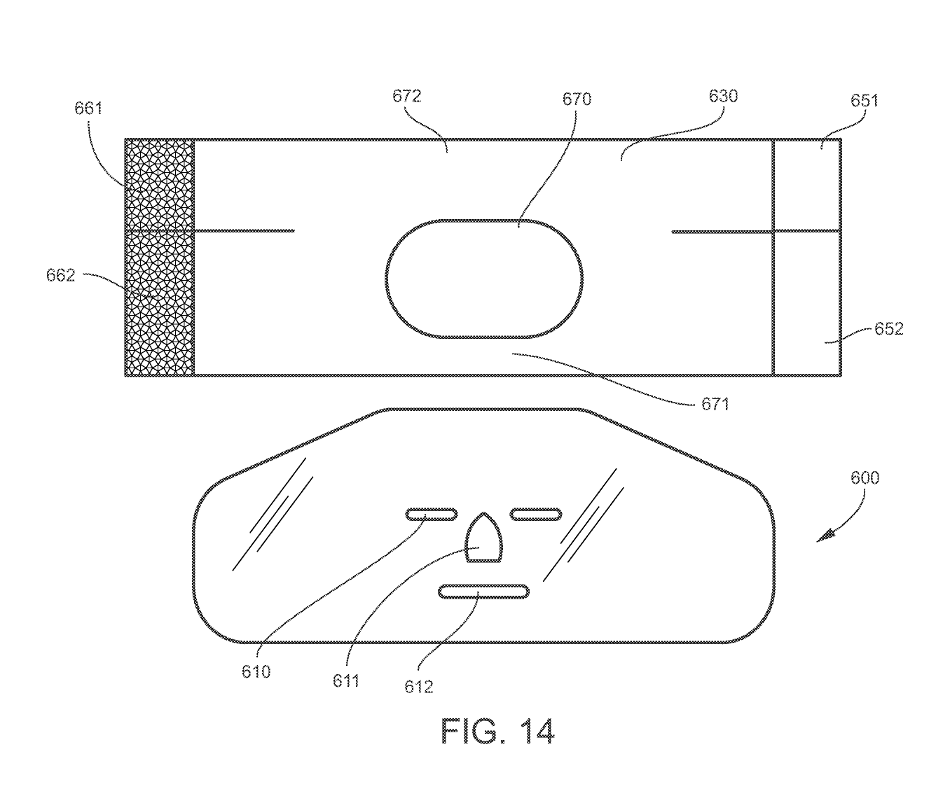

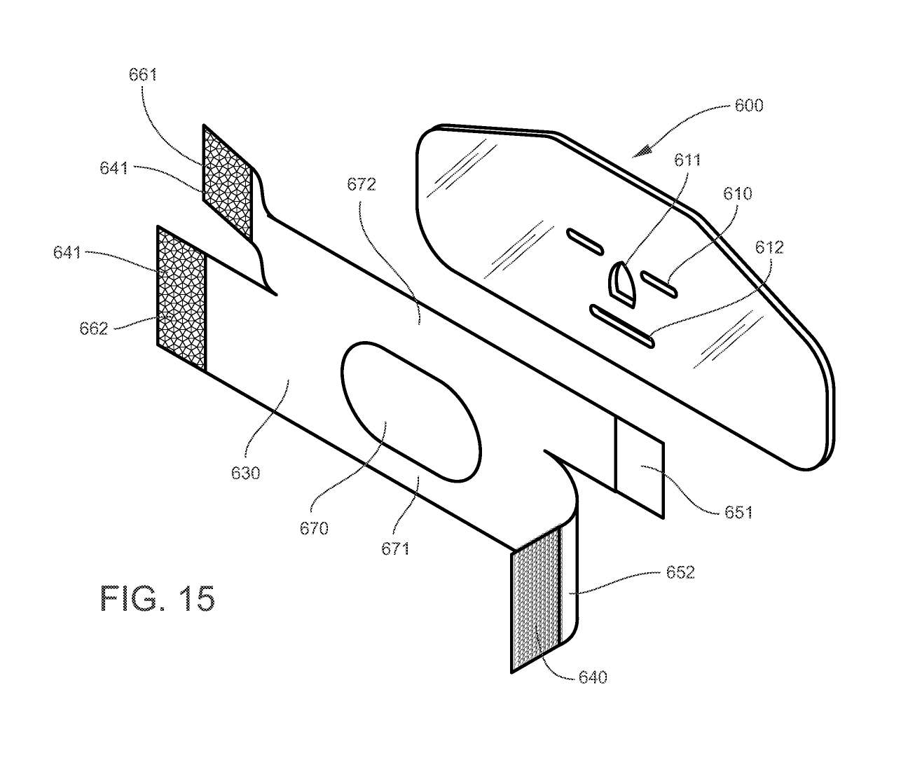

As further illustrated in FIGS. 14 and 15, in certain aspects, the strap 630 may have various alternative shapes and configurations. For example, the strap 630 may be bifurcated at both ends 651, 652, 661, 662. These bifurcated ends 651, 652, 661, 662 may further include complementary hook 640 and loop 641 fasteners. As further illustrated in FIGS. 14 and 15, the strap 630 may include a hole 670 positioned mid-span along the length of the strap 630 such that portions 671, 672 of the strap are configured to concurrently wrap around a wearer's chin and forehead respectively while not contacting and/or obstructing any of the wearer's nose, eyes, mouth, and combinations thereof. The mask depicted in FIGS. 14 and 15 may also include a styrene based gel mask 600 that is removable or permanently attached to strap 630. In certain aspects, the styrene based gel mask 600 and the strap 630 are two separate components that are not permanently attached to one another. In this aspect, the styrene based gel mask 600 may further include eyeholes 610, a nosehole 611, and mouth hole 612 such that the mask 600 can be placed on, for example, the wearer's face, and then the strap 630 including the hole can be positioned over the styrene based gel mask to secure the mask to the wearer. For example, the strap 630 may be placed on the wearer such that portions 672, 671 of the strap around the hole 670 secure the forehead and chin portions respectively of the styrene based gel to the wearer and then the strap may be fastened by the hook and loop fasteners positioned on the bifurcated ends 651, 652, 661, 662.

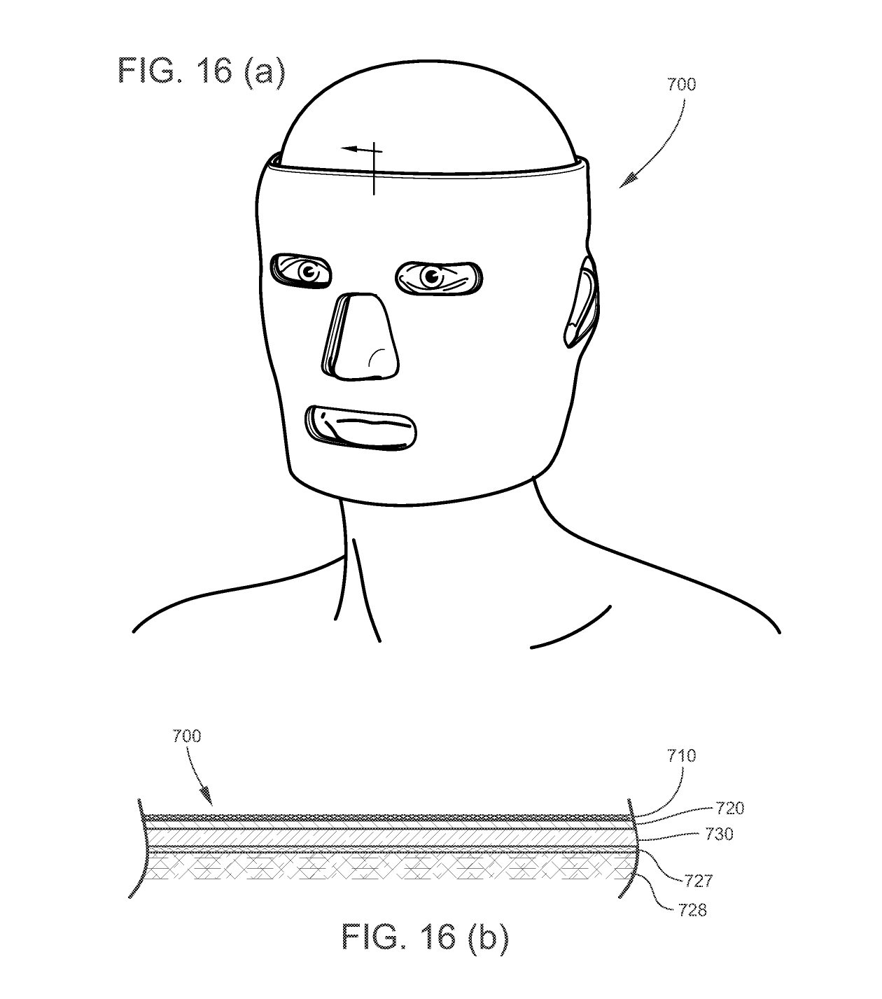

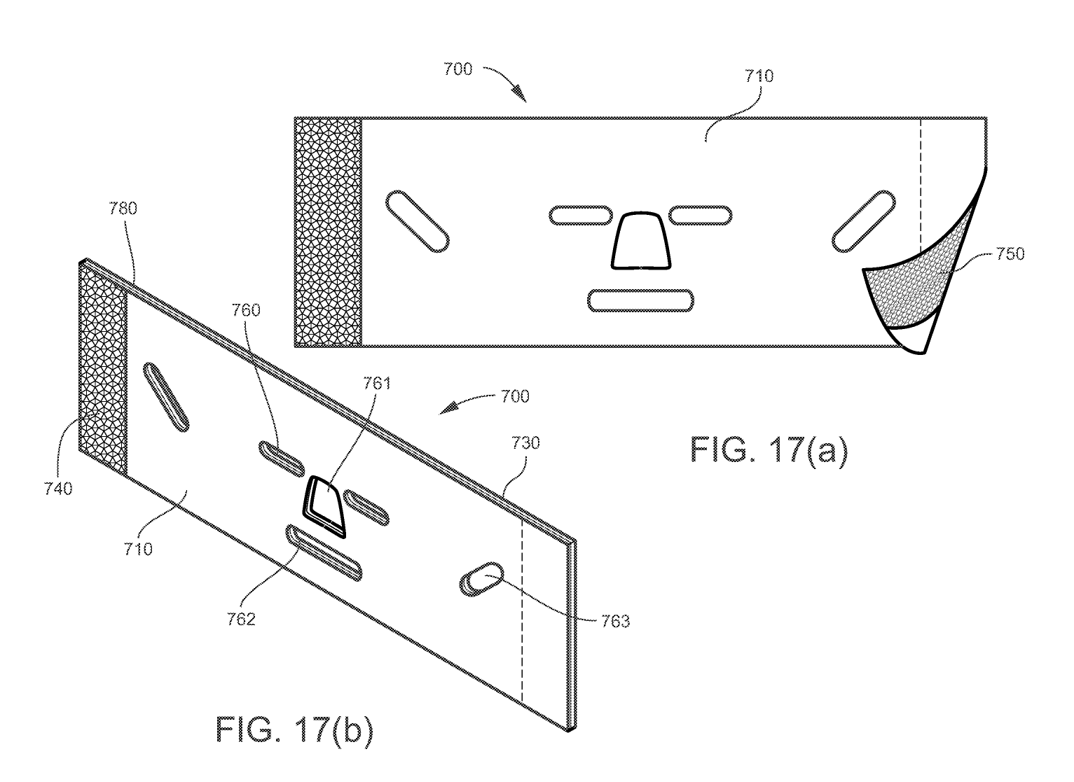

FIGS. 16(a), 16(b), 17(a), and 17(b) depict another embodiment of the post-surgical compression mask 700 in which the above discussed thermoformable assembly is included on or within an elongate strap 710 and is configured to apply compression. The strap 710 preferably includes sufficient elasticity to stretch over portions of the wearer's head (e.g., completely around the wearer's head), and the strap further preferably includes hook and loop fasteners 740, 750 positioned at opposite ends of the strap that preferably span the entire width the elongate strap 710. The elasticity of elongate strap 710 coupled with the hook and loop fastener widths allow for the compression mask to be properly secured to the wearer's head while concurrently ensuring maximum compression when compared to other conventional designs that lack elasticity and/or hook and loop fasteners having the above discussed features. Also, in this aspect, this compression mask 700 includes an outer knitted fabric layer 710 (i.e., portion of the elongate strap or the entire elongate strap) as disclosed above, a thermoformable resin 720 as disclosed above, and a styrene based gel 730 as disclosed above that is either detachably positioned or permanently positioned directly on the thermoformable resin 720. In certain aspects, this mask 700 includes eyeholes 760, a nosehole 761, a mouth hole 762, and optionally earholes 763. The styrene based gel 730 of this mask may be adapted to cover portions of the wearer's cheeks, chin, and forehead, and in some aspects, the styrene based gel 730 of this mask is adapted to additionally substantially cover portions of the wearer's head immediately adjacent either partially or completely around the wearer's ears. Similarly, the thermoformable resin 720 may also be adapted to cover portions of the wearer's cheeks, chin, and forehead, and in some aspects, the styrene based gel of this mask is adapted to additionally substantially cover portions of the wearer's head immediately adjacent either partially or completely around the wearer's ears. To maximize compression therapy results using this mask, the mask may be initially heated (e.g., to the thermoformable resin's glass transition temperature) as described above and the shaped to the contours of the wearer's face. After shaping this mask to the contours of the wearer's face, the mask may be allowed to harden to achieve a rigidly shaped structure and then subsequently secured onto the wearer's face using the hook and loop fasteners. If cold compression therapy or thermal therapy is further desired, the shaped mask may be chilled or heated to a sufficient temperature and the chilled or heated mask may be subsequently secured on the wearer's face. FIG. 16(b), depicts mask 700 in use in which gel layer 730 is placed on bandage 727, which directly covers flesh and/or post-surgical site 728.

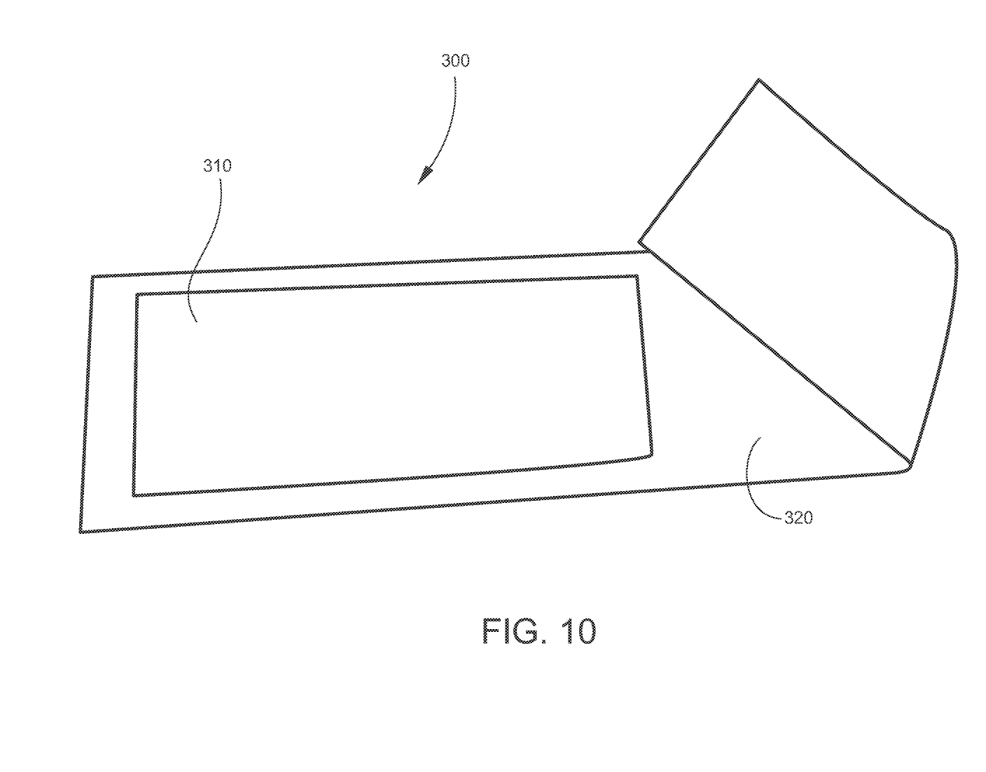

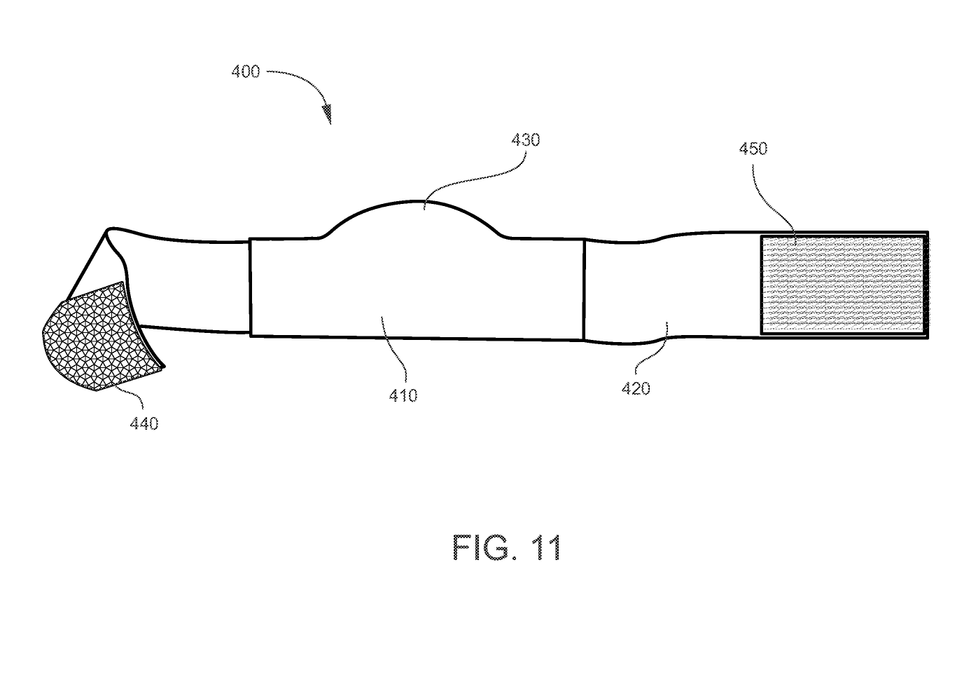

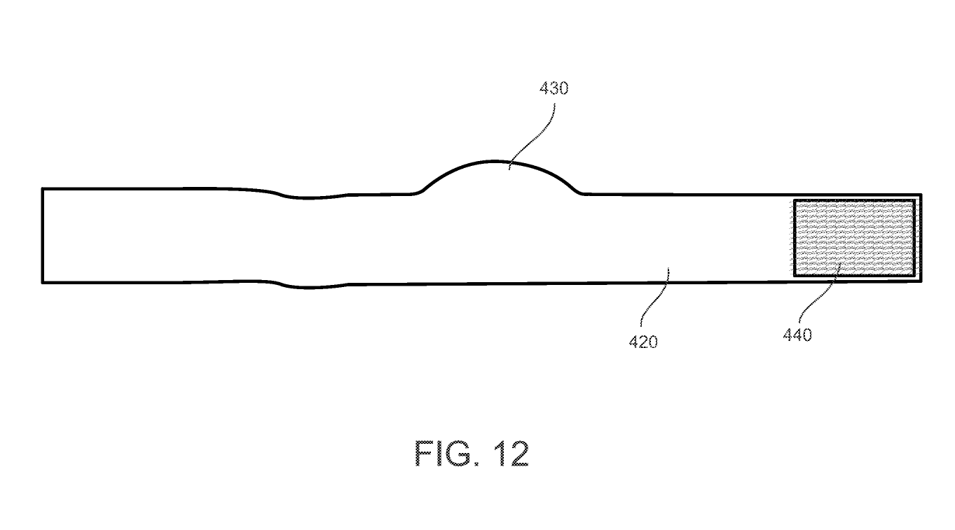

The garments disclosed herein may further include many different shapes and embodiments having a wide variety of uses. For example, these garments may be adapted for specific body parts and specific surgical procedures, which include, but are not limited, liposuction and surgical procedures involving the chin (e.g., chin implants), neck (e.g., a neck lift), etc. As shown in FIG. 10, another embodiment 300 may include the styrene based gel 310 permanently positioned on an elongate, elastic bandage 320 and used to apply post-surgical compression and/or for thermal therapy and/or cold compression therapy. In this aspect, a hook fastener (not shown) may be attached one end of the elongate, elastic bandage and may fasten directly to another portion of the elongate, elastic bandage. Alternatively, the elongate, elastic bandage may include complementary hook and loop fasteners (not shown) positioned on opposite ends of the elongate, elastic bandage such that a this garment may be securely fastened to a wearer. As further illustrated in FIGS. 11 and 12, another embodiment 400 may include the styrene based gel 410 being permanently positioned on an elongate, elastic bandage 420 and adapted for use on the chin, neck, or a combination thereof. In this aspect, FIGS. 11 and 12 particularly depict garment 400 useful, for example, in post-chin augmentation applications. The garment depicted in FIGS. 11 and 12 may include an arcuate shaped portion 430 of the gel and the elongate, elastic bandage adapted to cover a portion or substantially all of the wearer's chin.

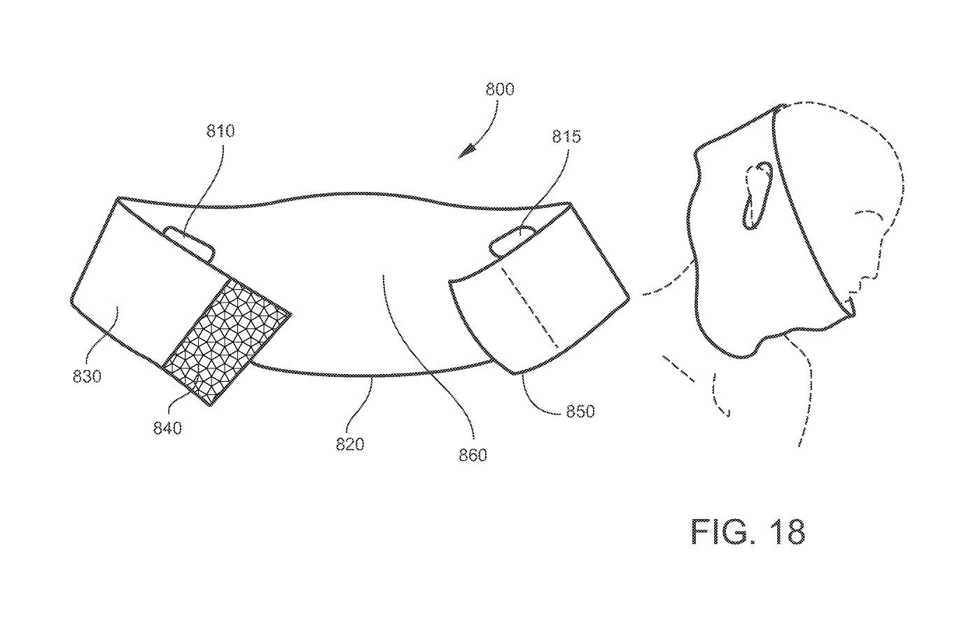

As shown in FIG. 18, in certain embodiments it is advantageous to provide a garment 800 having a substantial strength and sufficient width to provide robust neck and chin support post-surgical procedures involving the neck (e.g., a necklift, a necklift and chin augmentation, etc.). In this aspect, garment 800 is very similar to garment 400. For example, garment 800 includes the styrene based gel 820 positioned on an elongate, elastic bandage 830. Garment 800 further includes complimentary hook and loop fasteners 840, 850 positioned on opposing ends of garment 800. However, garment 800 further includes earholes 810, 815 from which a wearer's ears may protrude through when wearing garment 800. Garment 800 provides robust support to wearer's neck following post-surgical neck procedures. In addition, portions 860 of the garment positioned in between earholes 810, 815 have a greater width than (i) portions of the garment extending between earhole 810 and hook or loop fastener 840 and (ii) portions of the garment extending between earhole 815 and hook or loop fastener 840. In other words, portions of the garment extending between earhole 810 and hook or loop fastener 840 and portions of the garment extending between earhole 815 and hook or loop fastener 840 are tapered relative to the width of garment 860 positioned between earholes 810, 815. These earholes 810, 815 and tapered features may be particularly preferred to provide robust support of a wearer's neck that further aids and expedites healing of the neck and/or chin following surgical procedures.

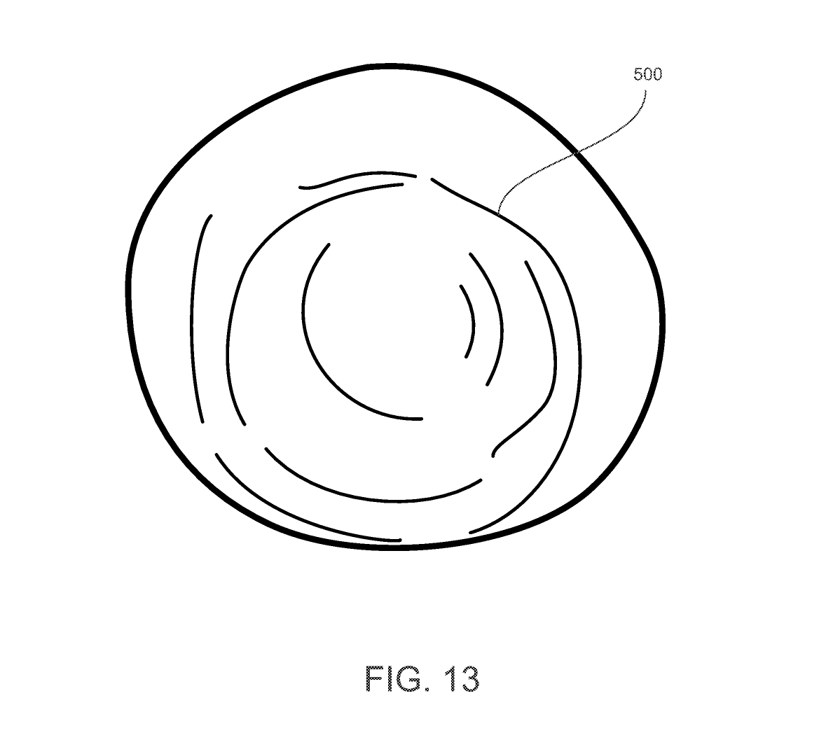

As illustrated in FIG. 13, in another embodiment, breast cup 500 includes the styrene-based polymeric gel adapted to conform to and cover a breast following a surgical procedure, which includes, but is not limited to breast augmentation, breast reconstruction, breast reduction, or combination thereof. In this aspect, the styrene-based polymeric gel may be molded (e.g., injection molded) to have the general shape and circumference of the breast. After placing this styrene-based polymeric gel over a user's breast, this gel may be further secured to a wearer with an elongate, elastic bandage being wrapped around the wearer's breast. In this aspect, the styrene-based polymeric gel may further include a thickness of 0.1 inches to 0.5 inches, 0.15 inches to 0.3 inches which advantageously ensures maximum reduction and/or prevention of post-surgical, excessive bruising, swelling, and edema associated with surgical procedures and further aids in the beneficial effects of thermal therapy and cold compression therapy. In certain aspects, breast cup 500 is packaged or included within a kit that further includes at least one elongate, elastic strap such as any of the above disclosed elongate, elastic straps, the strap being configured to hold breast cup 500 on a wearer's breast while applying adequate pressure to potentially reduce bruising, swelling, and/or edema at the surgical site.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

What is claimed is:

1. A post-surgical treatment mask, comprising:
(a) a thermoformable assembly comprising a thermoformable resin arranged on a knitted fabric layer, the thermoformable assembly configured to be heated to a glass transition temperature of the thermoformable resin such that the thermoformable assembly can be shaped to conform to contours of a user's face, and
(b) a styrene-based polymeric gel layer arranged on the thermoformable assembly.

2. The post-surgical treatment mask of claim 1, wherein the thermoformable resin comprises a low-melt co-polyester, a poly-caprolactone, or a combination thereof.

3. The post-surgical treatment mask of claim 1, wherein the thermoformable assembly is configured to be heated to a temperature of no more than 80° F. above the glass transition temperature of the thermoformable resin.

4. The post-surgical treatment mask of claim 1, wherein the thermoformable assembly is the thermoformable resin arranged between an inner and outer knitted fabric layer.

5. The post-surgical treatment mask of claim 4, wherein the inner and outer knitted fabric layers are the same.

6. The post-surgical treatment mask of claim 4, wherein the inner and outer knitted fabric layers are different.

7. The post-surgical treatment mask of claim 4, wherein the outer knitted fabric layer and the inner knitted fabric layer independently comprise knitted bodies comprising multifilament yarns, wherein:
the multifilament yarns are selected from the group consisting of a non-low melt polyester yarn, a non-low nylon yarn, a non-low polyproprylene yarn, a non-low melt polyethylene yarn, cotton yarn, wool yarn, and combinations thereof

8. The post-surgical treatment mask of claim 4, wherein the outer and inner knitted fabric layers independently have multidirectional stretch.

9. The post-surgical treatment mask of claim 1, wherein the styrene-based gel is removable from the thermoformable assembly.

10. The post-surgical treatment mask of claim 1, wherein the stryene-based gel is from 0.1 to 0.625 inches in thickness.

11. The post-surgical treatment mask of claim 1, wherein the styrene-based gel has a thermal conductivity of 0.05 to 3.00 W/mk.

12. The post-surgical treatment mask of claim 1, further comprising a strap configured to secure around a user's head to hold the mask in place on the user's face.

13. The post-surgical treatment mask of claim 12, further comprising hook and loop fasteners for securing the strap to the mask.

14. The post-surgical treatment mask of claim 1, further comprising two straps configured to secure around a user's head to hold the mask in place on the user's face.

15. The post-surgical treatment mask of claim 1, wherein the mask is a partial face mask.

16. A post-surgical face mask, comprising:
a mask made from a styrene-based gel shaped to conform to and cover a wearer's face including a chin, a nose, cheeks, forehead, and ears, the mask having openings at the wearer's eyes, nose, and mouth; and
a strap configured to fasten around the forehead and under the chin to secure the mask in place.

17. A post-surgical treatment mask, comprising:
(a) a thermoformable assembly comprising a thermoformable knit body arranged on a knitted fabric layer, the thermoformable assembly configured to be heated to a glass transition temperature of the thermoformable knit body such that the thermoformable assembly can be shaped to conform to contours of a user's face, and
(b) a styrene-based polymeric gel layer arranged on the thermoformable assembly.

18. The post-surgical treatment mask of claim 17, wherein the thermoformable knit body comprises a low-melt polyester yarn and polyester fiber.

19. The post-surgical treatment mask of claim 18, wherein the low melt-polyester yarn is a multifilament yarn.

20. The post-surgical treatment mask of claim 17, wherein the thermoformable knit body is comprised of multifilament yarn in which at least two of the yarns in the multifilament yarn have different melting temperatures.
